(19) United States
(12) Patent Application Publication
(10) Pub. No.: US 2017/0181702 A1
(43) Pub. Date: Jun. 29, 2017

(54) WRIST-TYPE BODY COMPOSITION MEASURING APPARATUS

(71) Applicant: SAMSUNG ELECTRONICS CO., LTD., Suwon-si (KR)

(72) Inventors: Young Jun KOH, Seoul (KR); Kak NAMKOONG, Seoul (KR); Yeol Ho LEE, Anyang-si (KR); Myoung Hoon JUNG, Bucheon-si (KR)

(73) Assignee: SAMSUNG ELECTRONICS CO., LTD., Suwon-si (KR)

(21) Appl. No.: 15/211,642

(22) Filed: Jul. 15, 2016

(30) Foreign Application Priority Data
Dec. 29, 2015 (KR) 10-2015-0188853

(51) Int. Cl. A61B 5/00; A61B 5/053

(52) U.S. Cl. CPC A61B 5/681; A61B 5/0537; A61B 5/7278; A61B 5/0022; A61B 5/742; A61B 2562/04

(57) ABSTRACT

A wrist-type body composition measuring apparatus is provided to measure body composition conveniently. The wrist-type body composition measuring apparatus includes a main body, a strap, first and second inner electrodes, and first and second outer electrodes. The main body includes a measurer configured to measure a body impedance of a user and an analyzer configured to analyze a body composition of the user based on the measured body impedance.

### CROSS-REFERENCE TO RELATED APPLICATION

This application claims priority from Korean Patent Application No. 10-2015-0188853, filed on Dec. 29, 2015 in the Korean Intellectual Property Office, the disclosure of which is incorporated herein by reference in its entirety.

### BACKGROUND

1. Field

Apparatuses and methods consistent with exemplary embodiments relate relates to measuring biometric information of a user by using an apparatus wearable on the user's wrist.

2. Description of the Related Art

In the past, body composition was generally measured in hospitals for the purpose of medical check-up. However, with the recent improvement in living standards, and a growing interest in health and diet, people are keen to measure body composition in their daily lives and monitor body composition changes.

Measuring body composition refers to quantitative measurement of individual elements of body composition, such as water, proteins, bones, fat, and the like, which form the human body.

As a method of measuring body composition, bioelectrical impedance analysis is commonly used, which is cheap and harmless to humans. In the bioelectrical impedance analysis, a weak electric current is applied to the human body to calculate an amount of body water, muscles, body fat, and the like by using a value of electric resistance, i.e. the electrical impedance, of the human body, and information including a user's stature, weight, age, gender, and the like.

### SUMMARY

One or more exemplary embodiments provide a wrist-type body composition measuring apparatus that may measure body composition conveniently while being carried.

According to an aspect of an exemplary embodiment, there is provided a wrist-type body composition measuring apparatus including: a main body including a measurer configured to measure a body impedance of a user and an analyzer configured to analyze a body composition of the user based on the measured body impedance; a first inner electrode and a second inner electrode which are disposed on a rear surface of the main body to be in contact with the user; and a first outer electrode and a second outer electrode which are provided on a front surface of the main body to be in contact with the user during measurement of the body composition, and which are provided to be apart from each other by a distance that allows the user to contact the first and second outer electrodes with a finger.

Upon applying a current through the first inner electrode and the first outer electrode, the measurer may measure a voltage between the second inner electrode and the second outer electrode to measure the body impedance.

The first and second outer electrodes may be arranged to face each other.

The first and second outer electrodes may be arranged symmetrical to each other.

One of the first and second outer electrodes may surround the other one of the first and second outer electrodes.

The first and second outer electrodes may have an identical center.

An arrangement direction of the first and second inner electrodes and an arrangement direction of the first and second outer electrodes may be perpendicular to a length direction of the main body.

An arrangement direction of the first and second inner electrodes and an arrangement direction of the first and second outer electrodes may be identical to the length direction of the main body.

One of a first arrangement direction of the first and second inner electrodes and a second arrangement of the first and second outer electrodes may be identical to the length direction of the strap, and the other may be perpendicular to the length direction of the main body.

Upper surfaces of the first and second outer electrodes may be disposed on an identical plane.

The main body may configured to perform predetermined functions when the first and second outer electrodes are pressed.

### BRIEF DESCRIPTION OF THE DRAWINGS

The above and/or other aspects will be more apparent by describing certain exemplary embodiments, with reference to the accompanying drawings, in which:

FIG. 1 is a diagram illustrating a general body composition measuring apparatus using bioelectrical impedance analysis.

FIG. 2 is a block diagram illustrating an example of a wrist-type body composition measuring apparatus.

FIG. 3 is a perspective view illustrating an example of a wrist-type body composition measuring apparatus.

FIG. 4 is a perspective view of the wrist-type body composition measuring apparatus illustrated in FIG. 3, as seen from the rear side.

FIG. 5 is a perspective view of another example of the wrist-type body composition measuring apparatus, as seen from the rear side.

FIG. 6 is a perspective view of another example of the wrist-type body composition measuring apparatus.

FIG. 7 is a perspective view of yet another example of the wrist-type body composition measuring apparatus.

FIG. 8 is a perspective view of still another example of the wrist-type body composition measuring apparatus.

FIG. 9 is a cross-sectional view of an enlarged portion of the wrist-type body composition measuring apparatus illustrated in FIG. 6.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

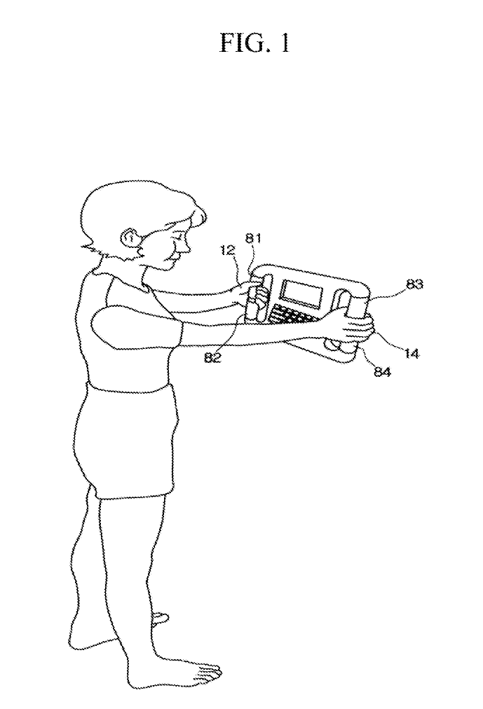

FIG. 1 is a diagram illustrating a body composition measuring apparatus using bioelectrical impedance analysis in the related art. The body composition measuring apparatus is designed to measure body composition with both hands 12 and 14 holding electrodes 81, 82, 83, and 84. However, while carrying the apparatus, it may be inconvenient to use the apparatus, since both hands are required to hold the electrodes.

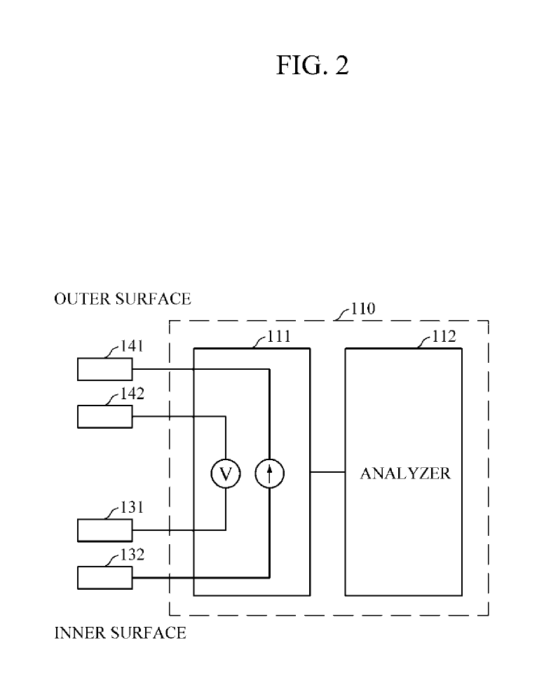

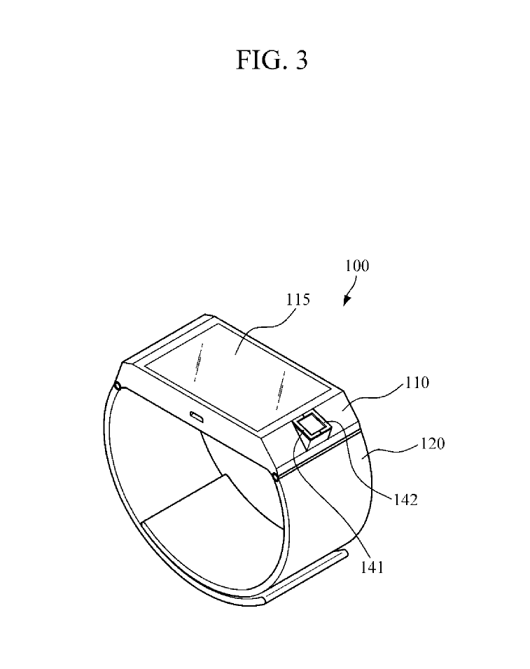

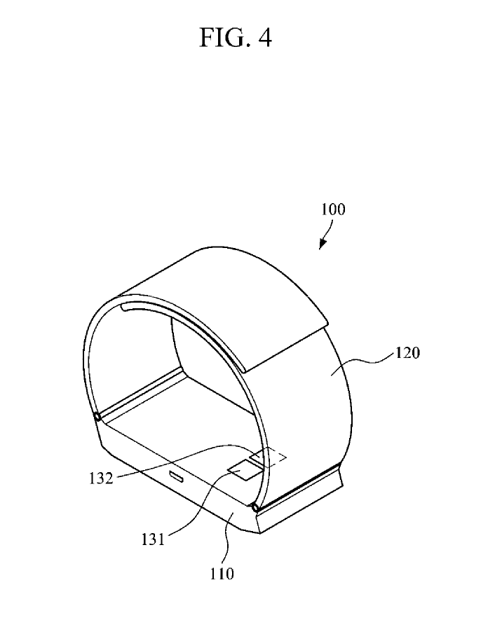

FIG. 2 illustrates a wrist-type body composition measuring apparatus 100 according to an exemplary embodiment. FIG. 3 is a perspective view illustrating the wrist-type body composition measuring apparatus 100, and FIG. 4 is a perspective view of the wrist-type body composition measuring apparatus 100 illustrated in FIG. 3, as seen from the rear side.

Referring to FIGS. 2 to 4, the wrist-type body composition measuring apparatus 100 includes a main body 110, a strap 120, a first inner electrode 131 and a second inner electrode 132, and a first outer electrode 141 and a second outer electrode 142.

The main body 110 includes a measurer 111 to measure body impedance and an analyzer 112 to analyze body composition of a user by using the body impedance measured by the measurer 111. Upon applying an alternating current between electrodes that are in contact with the body, the measurer 111 measures a voltage between the electrodes to calculate the body impedance. The alternating current may be a constant current of about 500 μ having a frequency of 50 kHz. Based on the measured body impedance, and stature, weight, age, and gender of a user, the analyzer 112 may calculate the total body water (TBW), body fat, and body fat percentage (%) using equations stored in the main body 110.

The strap 120 is connected to the main body 110, and is configured to be flexible. The strap 120 may be flexible enough to be wrapped around a user's wrist or may be unwrapped from the wrist, thereby enabling a user to put on or take off the wrist-type body composition measuring apparatus 100. The strap 120 may be made of urethane, silicone, rubber, leather, and the like.

The first and second inner electrodes 131 and 132 may be disposed on the rear surface of the main body 110 to be in direct contact with a user's body. That is, the first and second inner electrodes 131 and 132 may be in direct contact with a user's wrist. As illustrated in FIG. 4, the first and second inner electrodes 131 and 132 may be in a square shape. However, their shape is not limited thereto, and the first and second inner electrodes 131 and 132 may be in other shapes of, for example, a rectangle, a lozenge, a circle, a triangle, and the like.

As illustrated in FIG. 4, the first and second inner electrodes 131 and 132 may be arranged perpendicular to a length direction of the main body 110, but the arrangement of the first and second inner electrodes 131 and 132 are not limited thereto.

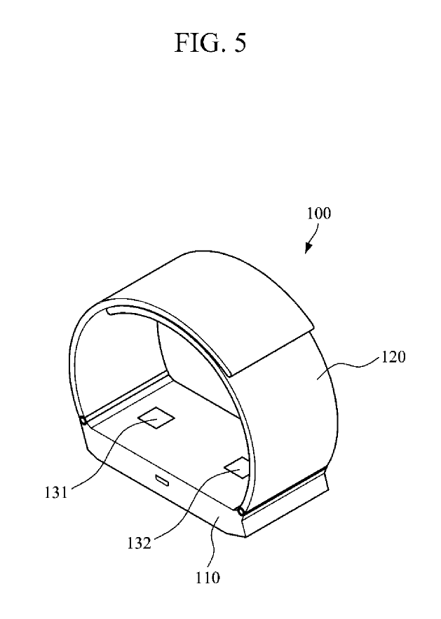

As shown in FIG. 5, the first inner electrode 131 and the second inner electrode 132 may be arranged to be apart from each other in the longitudinal direction of the main body 110.

The first and second outer electrodes 141 and 142 are disposed on the surface of the main body 110 to be in contact with a user during measurement of body composition, in which the first and second outer electrodes 141 and 142 are disposed close to each other to allow a user to contact both the first and second outer electrodes 141 and 142 with a finger. The second outer electrode 142 may have a circle shape and the first outer electrode 141 may have a ring shape that surrounds around the second outer electrode 142. The first and second outer electrodes 141 and 142 may have the same center point. As in the first and second inner electrodes 131 and 132, the first and second outer electrodes 141 and 142 may be arranged in a direction identical to the length direction of the strap 120, or may be arranged to face each other as illustrated in FIG. 3. Further, the first outer electrode 141 and the second outer electrode 142 may be disposed to face each other.

Once a user puts on the wrist-type body composition measuring apparatus 100 and touches the first and second outer electrodes 141 and 142 with a finger or a part of the palm, the measurer 111 applies an alternating current and measures a voltage to calculate body impedance, and the analyzer 112 analyzes body composition of the user by using the body impedance calculated by the measurer 111.

As described above, in the wrist-type body composition measuring apparatus 100, the first and second outer electrodes 141 and 142, which contact a user during measurement of body composition, are mounted on the surface of the main body 110, rather than on the strap 120, such that the strap 120 may be readily changed, and a degree of freedom of the disposition of electrodes and aesthetic impression may be increased. Further, the first and second outer electrodes 141 and 142 are disposed close to each other to allow a user to touch them with a finger, such that body composition may be measured with only a finger or a part of the palm, enabling convenient measurement of body composition.

Upon applying a current through the first inner electrode 131 and the first outer electrode 141, the measurer 111 measures the voltage between the second inner electrode 132 and the second outer electrode 142 to measure body impedance.

Specifically, while the first and second inner electrodes 131 and 132 are in contact with a wrist, the first and second outer electrodes 141 and 142 are contacted with a user's finger or a part of the palm, such that the first outer electrode 141 and the first inner electrode 131 form a closed circuit. Then, a sine wave alternating current is applied, and a voltage between the second outer electrode 142 and the second inner electrode 132 is measured, to calculate body impedance based on the applied current and the measured voltage.

As described above, in the case where body impedance is measured by using two inner electrodes and two outer electrodes, i.e., four electrodes, body impedance may be measured more accurately than the case where only two electrodes are used.

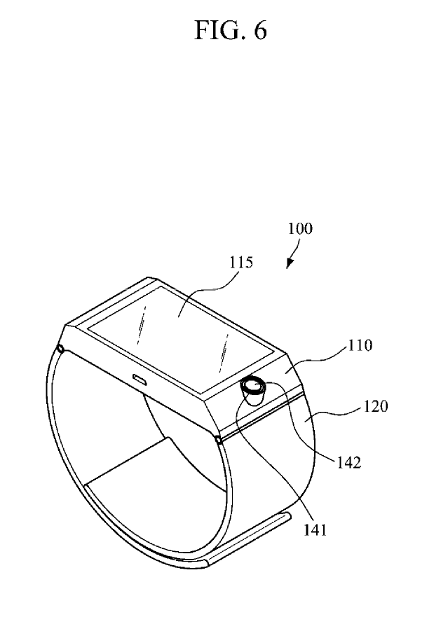

FIG. 6 is a perspective view of another example of the wrist-type body composition measuring apparatus 100.

Referring to FIG. 6, the first and second outer electrodes 141 and 142 may be formed in such a manner that one electrode 141 surrounds the other electrode 142. Here, the first outer electrode 141 surrounds the second outer electrode 142, but an opposite configuration may also be implemented. Further, the first and second outer electrodes 141 and 142 may be formed to have the same center.

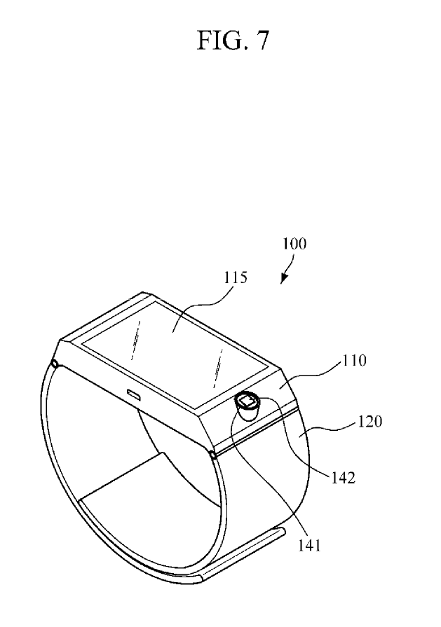

In another example, as in the wrist-type body composition measuring apparatus 100 illustrated in FIG. 7, one electrode 141 may surround the other electrode 142, and the two outer electrodes 141 and 142 may be formed to have the same center, in which the inner electrode may be in a square shape and the outer electrode may be in a circular shape. The inner electrode may be in a circular shape and the outer electrode may be in a square shape, and other shapes, rather than a square or circle, may also be applied.

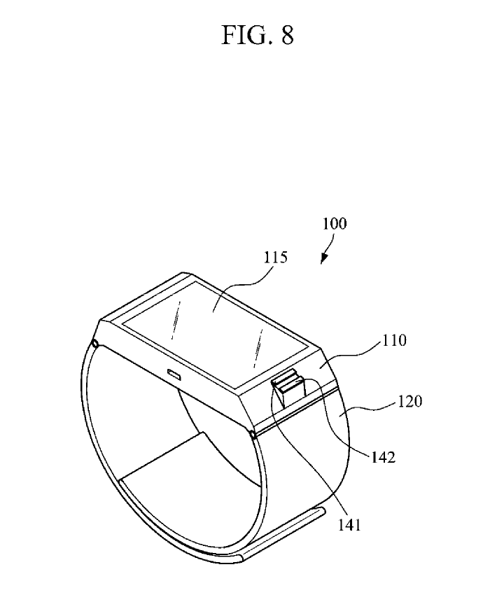

In addition, as in the wrist-type body composition measuring apparatus 100 illustrated in FIG. 8, the first and second outer electrodes 141 and 142 may be arranged symmetrical to each other. While FIG. 8 illustrates two symmetrical electrodes arranged in a direction identical to the length direction of the strap 120, the two symmetrical electrodes may be arranged perpendicular to the length direction of the strap 120, and may be in other shapes rather than a rectangular shape.

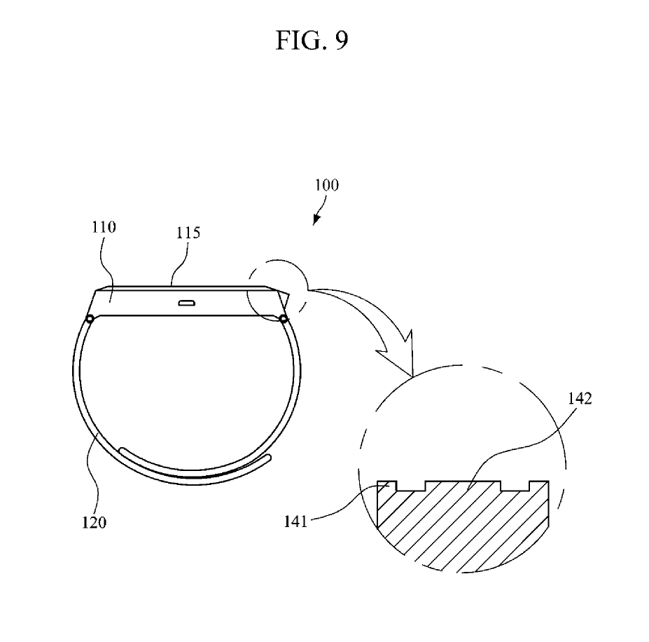

FIG. 9 is a cross-sectional view of the wrist-type body composition measuring apparatus 100 illustrated in FIG. 6. As illustrated in FIG. 9, upper surfaces of the first and second outer electrodes 141 and 142 may be formed on the same plane. In this case, when a finger or a part of the palm is contacted with the first and second outer electrodes 141 and 142, the contact may be made accurately.

In the aforementioned embodiments, the first and second outer electrodes 141 and 142 are formed on a lower front side of the main body 110, which is merely illustrative, and the first and second outer electrodes 141 and 142 may be formed on the upper side of the main body 110 or on the left or right side of the main body 110.

Further, the first and second outer electrodes 141 and 142 may be formed to be pressed, so as to operate as functional buttons that perform predetermined functions upon being pressed. That is, once the first and second outer electrodes are pressed, numbers are input or a screen is moved from side to side to enable a user to select a specific function.

Further, although not illustrated herein, the main body 110 may include a wireless communicator. The wireless communicator may include a Bluetooth module, a Radio Frequency (RF) module, and the like. The wireless communicator may transmit body composition information, analyzed by the analyzer 112, to a smartphone and the like, thus enabling a user to check and manage the body composition information on their smartphone or the like.

The main body 110 may include a display 115. The display 15 is mounted in such a manner that a screen of the display 115 may be exposed to the outside of the main body 110. The display 115 displays the body composition information, analyzed by the analyzer 112, in numbers or characters to show the information to a user.

The wrist-type body composition measuring apparatus may measure body composition conveniently while being carried. Further, the wrist-type body composition measuring apparatus may increase a degree of freedom of the disposition of electrodes and aesthetic impression. In addition, the wrist-type body composition measuring apparatus may conveniently measure body composition by using only one finger or a part of the palm.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A wrist-type body composition measuring apparatus comprising:
a main body comprising a measurer configured to measure a body impedance of a user, and an analyzer configured to analyze a body composition of the user based on the measured body impedance;
a first inner electrode and a second inner electrode which are provided on a rear surface of the main body to be in contact with the user; and
a first outer electrode and a second outer electrode which are provided on a front surface of the main body to be in contact with the user during measurement of the body composition, and which are disposed to be apart from each other by a distance that allows the user to contact the first and second outer electrodes with a finger.

2. The apparatus of claim 1, wherein upon applying a current through the first inner electrode and the first outer electrode, the measurer is further configured to measure a voltage between the second inner electrode and the second outer electrode to measure the body impedance.

3. The apparatus of claim 1, wherein the first and second outer electrodes are arranged to face each other.

4. The apparatus of claim 2, wherein the first and second outer electrodes are arranged symmetrical to each other.

5. The apparatus of claim 1, wherein one of the first and second outer electrodes surrounds the other one of the first and second outer electrodes.

6. The apparatus of claim 5, wherein the first and second outer electrodes are have an identical center.

7. The apparatus of claim 1, wherein an arrangement direction of the first and second inner electrodes and an arrangement direction of the first and second outer electrodes are perpendicular to a length direction of the main body.

8. The apparatus of claim 1, wherein an arrangement direction of the first and second inner electrodes and an arrangement direction of the first and second outer electrodes are identical to a length direction of the main body.

9. The apparatus of claim 1, wherein one of a first arrangement direction of the first and second inner electrodes and a second arrangement direction of the first and second outer electrodes is identical to a length direction of the main body, and the other is perpendicular to the length direction of the main body.

10. The apparatus of claim 1, wherein upper surfaces of the first and second outer electrodes are disposed on an identical plane.

11. The apparatus of claim 1, wherein the main body is configured to perform predetermined functions when the first and second outer electrodes are pressed.
